(19) United States
(12) Patent Application Publication
(10) Pub. No.: US 2013/0060152 A1
(43) Pub. Date: Mar. 7, 2013

(54) APPARATUS AND METHOD FOR CONTINUOUS OSCILLOMETRIC BLOOD PRESSURE MEASUREMENT

(71) Applicant: Ehud Baron, Tokyo (JP)

(72) Inventor: Ehud Baron, Tokyo (JP)

(73) Assignee: CARDIOSTAR, INC., Mentor, OH (US)

(21) Appl. No.: 13/643,596

(22) PCT Filed: Apr. 28, 2011
(86) PCT No.: PCT/IB11/00921

§ 371 Date: Oct. 26, 2012

Related U.S. Application Data
(60) Provisional application No. 61/329,086, filed on Apr. 28, 2010.

(51) Int. Cl. A61B 5/0225; A61B 5/025; A61B 5/022

(52) U.S. Cl. 600/494; 600/499; 600/495

(57) ABSTRACT

A method that extends the oscillometric method, which currently is used for blood pressure measurement at one point in time, to provide continuous measurement of blood pressure (BP). The method provides a BP signal that is similar to an invasive arterial line continuous BP measurement with minimal changes in clinical procedures. The apparatus for performing the method includes a sensor with a fluid-filled, disposable, flexible bladder underneath a non-invasive inflatable cuff monitor. The inflatable cuff monitor provides a single-point BP value in a traditional manner. An electronic scaling adapter estimates the diastolic pressure and systolic pressure corresponding to the BP signal obtained from the fluid-filled bladder, uses the single-point BP value from the inflatable cuff monitor to scale the BP signal detected by the bladder, and outputs a scaled BP signal that can be displayed from a conventional vital signs monitor.

### RELATED APPLICATIONS

This application claims the benefit of U.S. Provisional Patent Application Ser. No. 61/329,086, filed Apr. 28, 2010, which is hereby incorporated herein in its entirety by this reference.

### FIELD OF THE INVENTION

The present invention relates to an apparatus and method for blood pressure monitoring, and in particular to an apparatus and method for continuous, non-invasive blood pressure monitoring.

### BACKGROUND

In critical care medical situations blood pressure (BP) is usually monitored continuously, using an invasive, fluid-filled monitoring line, also called an arterial line or an A-line. A catheter is inserted into an artery and blood pressure from the artery is transmitted to a blood pressure transducer through fluid-filled tubing connected between the catheter and the transducer. Unfortunately, this invasive method of monitoring blood pressure is associated with high risks of complications, such as infection, thrombosis, and air embolism, and requires continuous delivery of anti-coagulant, for example. Consequently, non-invasive measurement methods that provide continuous blood pressure monitoring are advantageous as they avoid many of the complications associated with invasive monitoring.

When an A-line cannot be used, the only other clinically-accepted alternative to an invasive A-line is a non-continuous blood pressure measurement using the principle of oscillometric blood pressure measurements. This approach includes: (1) inflating an air cuff around the wrist or arm to a pressure above the systolic blood pressure (which causes arterial occlusion), and then (2) releasing the pressure and measuring the pulse signal amplitude during the pressure decay. In an automated system, a third step (3) includes determining the mean arterial pressure as the maximum pulse signal and calculating the empirically-accepted systolic and diastolic blood pressures. Some of these automated systems are referred to as non-invasive blood pressure (NIBP) systems, but not all non-invasive blood pressure monitoring systems that are called NIBP systems operate in the same way. In a manual system, the third step (3) includes listening for the distinct pulse sounds that characterize the systolic and diastolic pressures (a manual method commonly employing a manually-actuated air-pressurized cuff with a pressure gauge and a stethoscope). This manual method also is referred to as the auscultatory method.

Widely available automated NIBP systems include a standalone system (e.g., a wrist cuff device typically for home use) and a system that can be connected to a vital sign patient monitor (e.g., an arm cuff device typically used in a hospital setting). These devices provide BP measurements only at discrete points in time and cannot monitor the BP continuously between oscillometric measurements, which can result in missing serious BP events between measurements.

Although attempts have been made to provide devices that can perform continuous non-invasive BP monitoring, none have been clinically accepted. These devices have either employed technology that is substantially different from the oscillometric method, such as that using tonometry or have employed blood pressure measurements at very high pressures, typically on a finger (the wrist and arm generally cannot endure continuous high pressures without the limb becoming ischemic). Some of these systems are described in U.S. Pat. Nos. 4,869,261; 4,660,544; 4,343,314; 6,589,185; and 7,041,060, for example. In addition to various BP measurement technologies, some other patents describe an apparatus for interfacing a continuous BP signal to the A-line input of a patient vital sign monitor, such as U.S. Pat. Nos. 5,605,156; 6,176,831; 6,471,646; and 7,503,897.

### SUMMARY

One problem with previous automated non-invasive BP monitoring devices is that they rely on unproven technologies that deviate from the proven. While the oscillometric method uses a pressure cuff with certain proportions between circumference and width, the current devices that attempt to monitor BP non-invasively, like tonometry, use sensors placed on the patient next to the artery, in physical contact with the patient in proximity to the artery. Because the measurement technology is so different from the time-honored, clinician-accepted oscillometric method, in addition to the expensive nature of these products, current non-invasive BP monitors generally are not in routine clinical use. These products often introduce new measurement methods for non-invasive continuous BP monitoring by using new sensors and assemblies or new ways tp measure blood pressure. By doing so, they ignore the major value of many years of experience gained in the medical world using the oscillometric method, which is based on an air-inflatable pressure cuff.

The apparatus and method provided in accordance with the invention extends the accepted oscillometric method, used since 1876, to include continuous monitoring rather than just intermittent monitoring. The usage protocol is very similar to using an A-line, and thus medical professionals do not have to be re-educated before employing the non-invasive BP monitoring methods described herein.

More particularly, the present invention provides a non-invasive blood pressure apparatus for use with an air-pressurizable cuff that provides a near-continuous blood pressure (BP) signal output, the apparatus comprises: a fluid-filled bladder positionable under an air-pressurizable cuff, a pressure transducer coupled to the bladder through a fluid-filled line, and a controller in electrical communication with the transducer to translate nearly continuously the pressure signal from the transducer into a BP value. The apparatus can further comprise a monitor and means for inputting a BP value determined from the oscillometric method using an air-pressurizable cuff, the controller being configured to scale and output the near-continuous BP signal to the oscillometrically-measured BP in real time.

The apparatus also can include an air-pressurizable cuff and a transducer coupled to the cuff to output the air pressure in the cuff to the controller. The controller is connected to a pump and the air-pressure transducer to monitor and control the pressure in the air cuff so that the air-pressure transducer signal during an inflation phase can be used to measure BP, which provides an ability to measure at least one of systolic, mean arterial pressure (MAP), and diastolic pressure in either an inflation phase or a deflation phase. The air-pressurizable cuff may be provided by a commercially-available NIBP device.

The present invention also provides a non-invasive blood pressure apparatus that includes a controller configured to manipulate a pressure at which a sensing bladder is pressed against an artery, where the controller includes computational means configured to determine at least one of a systolic and a diastolic pressure and to scale a blood pressure signal accordingly.

The present invention also provides a controller configured to connect between a fluid-filled bladder that can be placed on a patient's skin in proximity to an artery, a pressure sensor, and a vital signs monitor. The controller includes: (i) a first input port configured to receive a signal indicative of a BP signal of a subject; (ii) a processor configured to receive the signal and to control a fluid pump to manipulate bladder pressure and determine diastolic and systolic BP and to scale an output signal indicative of the BP of the subject patient according to a predetermined algorithm based on the oscillometric method; and (iii) an output port configured to provide the output signal in a form suitable for input to a monitor. The controller preferably is configured to enable a standard vital signs monitor to display the scaled output signals. The present invention also provides for use of the apparatus for computing derived hemodynamic parameters like cardiac output, central BP, and systemic vascular resistance in a continuous way.

In addition to the above, the present invention provides a method for calculating a blood pressure of a subject by manipulating the pressure of a fluid-filled bladder placed on a patient's skin in proximity to a palpable artery. The method includes the following steps: a) increasing bladder pressure and measuring a relationship between pulse amplitude and pressure change; and b) changing the bladder pressure in a periodic manner and estimating from a change in pulse amplitude and shape the mean arterial pressure, diastolic pressure, and systolic pressure.

The present invention further provides an oscillometric BP measurement device that sweeps cuff pressure around a predetermined value below mean arterial pressure to obtain continuous measurement of the MAP. The device can use incrementally larger cuff pressure sweeps to estimate a shape of oscillatory pulse distribution. The present invention further provides an oscillometric BP measurement device that sweeps cuff pressure around a predetermined value below mean arterial pressure to estimate systolic and diastolic BP.

The foregoing and other features of the invention are hereinafter fully described and particularly pointed out in the claims, the following description and annexed drawings setting forth in detail certain illustrative embodiments of the invention, these embodiments being indicative, however, of but a few of the various ways in which the principles of the invention may be employed.

### BRIEF DESCRIPTION OF THE DRAWINGS

For a better understanding of the invention and to show how it may be carried into effect, reference will now be made, purely by way of example, to the accompanying drawings.

With specific reference now to the drawings in detail, the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention; the description taken with the drawings making apparent to those skilled in the art how the several forms of the invention may be embodied in practice. In the accompanying drawings:

FIG. 1 depicts the pulse amplitudes as a function of pressure decay for the oscillometric BP method. The bottom figure presents the pulse pressure amplitudes after the background pressure decay has been subtracted. The maximum pulse amplitude corresponds to the mean arterial pressure (MAP). The systolic and diastolic pressures are an accepted percentage to the left and right of the MAP, respectively.

FIG. 2 describes the schematic structure of a PCM-50 continuous BP monitor provided in accordance with the invention.

FIG. 3 shows results of the arterial pulse pressure readings for seven oscillometric measurements, one every 3 minutes (top) and an expanded view over an oscillometric measurement (bottom) after subtraction of the oscillometric pressure decay.

FIG. 4 depicts the BP signal around the mean arterial pressure and the change in the pulse waveform as result of changes in external pressure.

FIGS. 5 and 5A depict the change in the BP signal shape when the external pressure is above the systolic pressure (FIG. 5), compared to the shape of the BP signal when the external pressure is below the diastolic pressure (FIG. 5A).

FIG. 6 is a schematic presentation of a PCM-100 disclosed apparatus provided in accordance with the invention.

FIG. 7 shows the changes in the signals produced by the scaling adapter, in order to emulate an A-line to a standard monitor.

FIG. 8 illustrates wavelets analysis and how it is employed for removing noise in BP signal wave analysis.

FIG. 9 further illustrates the signal processing using wavelets analysis and how it is employed for removing noise in BP signal wave analysis.

FIG. 10 shows the use of a Kalman filter control algorithm to facilitate tracking of MAP, diastolic and systolic BP changes between two oscillometric measurements from a pressure cuff.

FIG. 11 illustrates how varying the external pressure around a predetermined pressure, generally below the mean arterial pressure, can help to track the MAP and scale the measured BP signal to its real values.

FIG. 12 shows the use of a fuzzy logic control algorithm to facilitate tracking of MAP, diastolic and systolic BP changes between two oscillometric measurements from a pressure cuff.

### DETAILED DESCRIPTION

A better understanding of the disclosed invention can be gained from the following detailed explanation of the figures, which represent, for illustrative purposes only, a particular embodiment. The scope of the present invention includes both combinations and sub-combinations of the various features described herein, as well as variations and modifications thereof which would occur to persons skilled in the art upon reading the following description.

There is a growing need for non-invasive continuous blood pressure (BP) monitoring for diagnosis and treatment assessment of cardiovascular diseases. As stated above, doctors and other health professionals have used a non-invasive BP measurement for many years using an inflatable pressure cuff and the oscillometric method. The oscillometric method is based on monitoring peak oscillatory vibrations when the limb is pressed by an inflated cuff with certain dimensions. The disclosed invention extends the oscillometric principle from one-time BP measurements to continuous measurements, without the need to completely occlude the arterial flow. In addition, it provides continuous beat-to-beat blood pressure readings with high fidelity in the detected pulse waveform. Unlike previous attempts, the system provided by the invention follows the widely-accepted oscillometric paradigm, and extends it to the continuous monitoring.

The embodiments of the disclosed invention described here solve one or more of the problems indicated above by adopting the following goals:

a. following the widely accepted oscillometric paradigm;

b. employing low cost disposable components where necessary;

c. employing low cost reusable components where possible;

d. providing a system that does not require extensive training to use; and

e. enabling the use of existing monitors and other standard components whenever possible.

The following description will describe two exemplary embodiments, designated PCM-50 and PCM-100, respectively. PCM stands for Personal Cardiovascular Monitor.

### PCM-50

The PCM-50 embodiment consists of a fluid-filled bladder that is placed above an artery, typically in the wrist, and underneath an independent air-pressurized cuff. The pressure cuff holds the fluid-filled bladder against a palpable artery. The pressure cuff can be provided in the form of a cornmercially-available NIBP device. The fluid-filled bladder communicates through fluid-filled tubing, preferably using an incompressible fluid, with a conventional A-line pressure transducer, although other types of sensors could be used in place of or in addition to an A-line pressure transducer. For purposes of this description, we will refer to the pressure sensor generically as a transducer, but the invention is not limited to an apparatus that uses a transducer. The transducer is connected electrically through a scaling adapter to the input of a monitor or other output device. The scaling adapter uses signal processing techniques to produce a BP signal which is continuous, and can be calibrated to the measured BP in mmHg units. The scaling adapter is an electronic device, generically referred to as a controller, that typically includes a memory, a processor, and the necessary inputs and outputs for accomplishing the tasks described.

An advantage of this embodiment is that an independent, automated non-invasive BP (NIBP) measurement device can be used as the air cuff (e.g., a wrist cuff), surrounding the continuous, non-invasive, fluid-filled bladder. The independent BP measurement device can be commercially-available NIBP device, as mentioned above, unaltered in any way. It continues to function in its intended manner, providing a measured blood pressure value for a single point in time. The independent BP measurement device is actuated to perform the oscillometric measurement. The fluid-filled bladder senses the pressure change created by the inflating pressure cuff and initiates a simultaneous oscillometric measurement. The scaling adapter monitors the bladder's pressure through the fluid-pressure transducer, in which the pressure is composed of both the air cuff deflation pressure as well as the pulse signal amplitude detected by the fluid-filled bladder. After the deflation of the air cuff, the presence of the cuff generally maintains sufficient pressure to hold the bladder against the artery to continue to monitor the pulse signal.

The scaling adapter uses the air pressure signal from the independent BP measurement device as a function of the air cuff's pressure decay to calculate the maximum pulse amplitude—the mean arterial pressure (MAP)—and the corresponding systolic and diastolic BP. Alternatively, the user can manually input the single-point BP value measured by the independent BP measurement device into the scaling adapter, which can then calculate the systolic and diastolic BP. These values are then used to scale the pulse amplitude that the fluid-filled bladder monitors between oscillometric measurements, when the pressure in the air cuff is low.

An air-pressure transducer or other pressure sensor can be incorporated into the independent BP measurement device or provided separately to detect the pressure in the air cuff. The air-pressure transducer can then output an independent oscillometric BP measurement that can be used to calibrate or to verify the BP determined from the bladder pressure signals. The oscillometric BP measurement can be repeated as necessary, for example, every fifteen minutes to ensure that the scaled BP values obtained from the fluid-filled bladder remain accurate.

In addition to the oscillometric BP measurement from pressure in the air cuff and the BP monitoring of the scaled pulse signal from the pressure in the bladder between these oscillometric measurements, the scaling adapter can also be configured to track changes in the BP during the monitoring period. The scaling adapter can perform this BP systolic and diastolic value adjustment by monitoring the change in the pulse signal amplitude and shape and correlating this information to a new systolic and diastolic BP from the pressure decay curve obtained in a previous oscillometric measurement. The scaling adapter also can have alert limits incorporated into or manually inputted to detect gross changes in the pulse signal between the oscillometric measurements. The scaling adapter also could include a means to control the pressurization of the fluid-filled bladder, such as with an incorporated pump (e.g., syringe pump) in fluid communication between the bladder and a fluid reservoir.

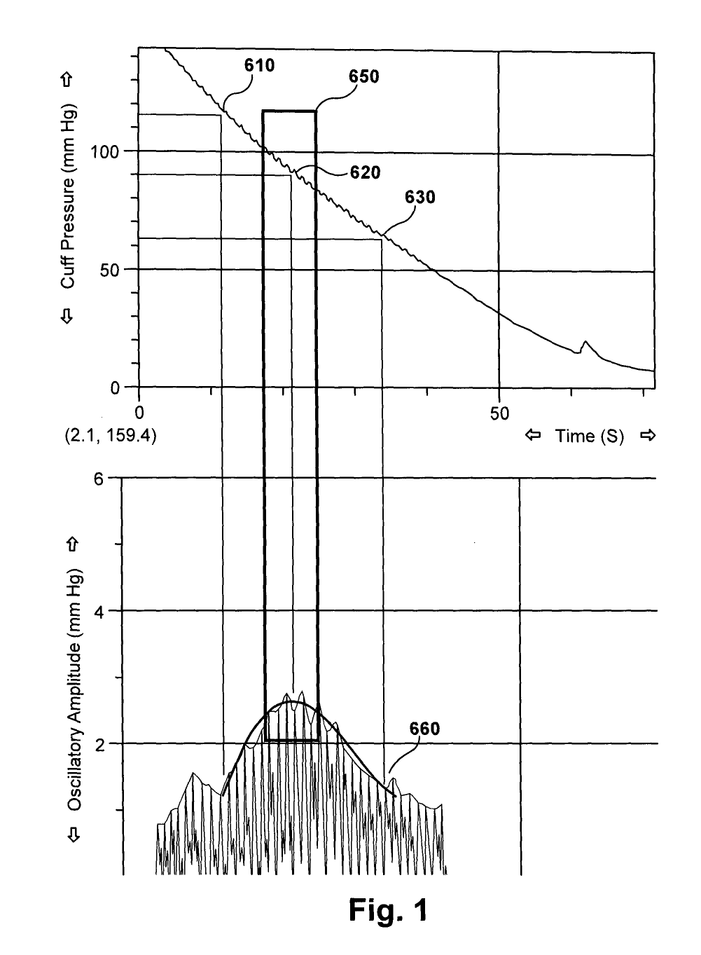

FIG. 1 depicts the oscillometric method of measurement of BP. It is based on an observation made more than 100 years ago that the pulse vibration when de-trended from the pressure decay curve, reaches a maximum amplitude when the mean arterial pressure (MAP) 620 is reached. Systolic 610 and diastolic 630 pressures are estimated from the vibration distribution 660 where the systolic BP corresponds to the point of 60% of the maximum amplitude above the MAP and the diastolic BP corresponds to 80% of the maximum amplitude below the MAP. A narrow pressure decay window around the MAP is designated by the rectangular box 650.

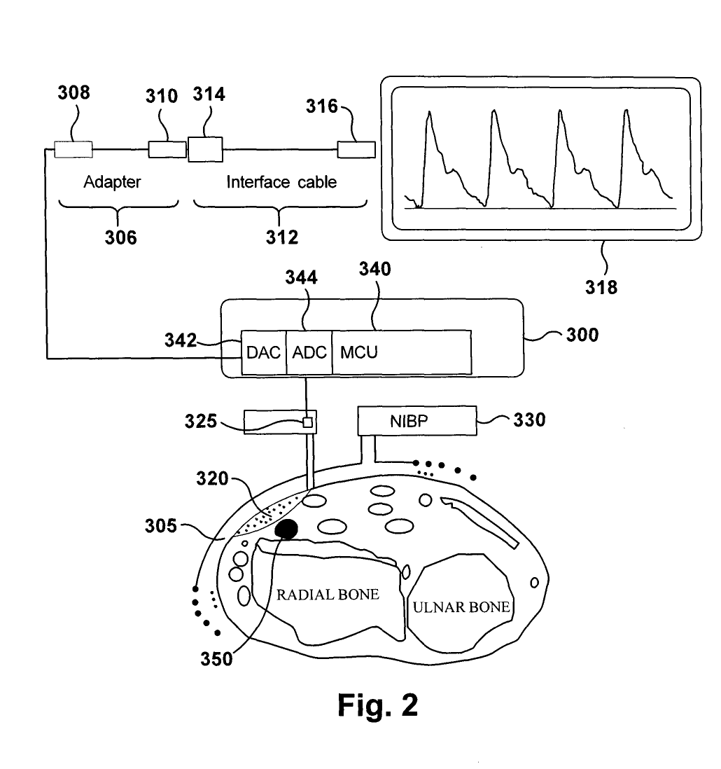

FIG. 2 depicts the structure of the PCM-50 embodiment. In this embodiment, the PCM-50 includes a disposable bladder 320 that can be filled with fluid, generally an incompressible liquid, and an air-pressurized cuff 305 that can be placed around a limb of a patient, typically near an upper arm or wrist. The system also includes a scaling adapter controller 300 electrically connected to a pressure transducer 325, such as a conventional A-line transducer. The transducer 325 is coupled to the bladder 320 through a fluid-filled tube, similar to that used in an A-line system. The scaling adapter controller also can control the pressure in the bladder by controlling a small pump that is connected between a fluid reservoir and the bladder (not shown).

In use, the bladder 320 is sandwiched between the patient and the pressure cuff 305, adjacent an artery 350, in this example the radial artery. The bladder, fluid, and transducer cooperate to detect the patient's pulse signals and convert it into an electrical signal that is transmitted to the scaling adapter 300. The scaling adapter computes the systolic pressure, the mean arterial pressure, and the diastolic pressure, and scales the output pulse signal accordingly. The scaling adapter then transmits or otherwise outputs an output signal that can be used to display the calibrated BP signal, such as on a vital signs monitoring device 318.

In FIG. 2, the pressure in the air cuff is controlled by its own controller 330, which operates its air pump, pumping air into the cuff 305 independently of the pressure in the fluid-filled bladder. In the PCM-50 system, the fluid-filled bladder continuously measures both the pressure in the air cuff and the arterial pulse signal, and relies on the air cuff to provide the pressurization for the oscillometric measurement. The bladder transducer signal is converted to BP values by scaling the pulse amplitudes with the values determined in the oscillometric method. In addition, depending on the output device, the BP signal can then be further adjusted (e.g., digital-to-analog) for compatibility with a monitor or other output device.

The scaling adapter 300 can be connected to a standard A-line monitor through an adapter 306 with connectors 308 and 310. The adapter 306 can be connected to the monitor 318 by a cable 312 with connectors 314 and 316.

The scaling adapter can emulate an A-line transducer in such a way that an A-line monitor sees the scaling adapter output as if it were a regular A-line transducer connected to an invasive fluid-filled arterial line. The scaling adapter can be designed to detect the excitation voltage supplied by the monitor and for any given measured blood pressure, output an equivalent A-line transducer output signal. Thus the signal corresponds to a signal that would have been produced for that pressure by a transducer with which the A-line monitor is configured to work. For example, for a transducer sensitivity of 5 μV/V/mmHg, an excitation voltage of 5 V and a pressure of 100 mmHg, an A-line transducer will output a differential voltage of (5 μV/V/mmHg)*(5 V)*(100 mmHg), or 2.5 mV. For the same combination of sensitivity, excitation voltage and pressure, the scaling adapter will also output the same differential voltage of 2.5 mV. The scaling adapter is connected between the bladder transducer and the A-line monitor.

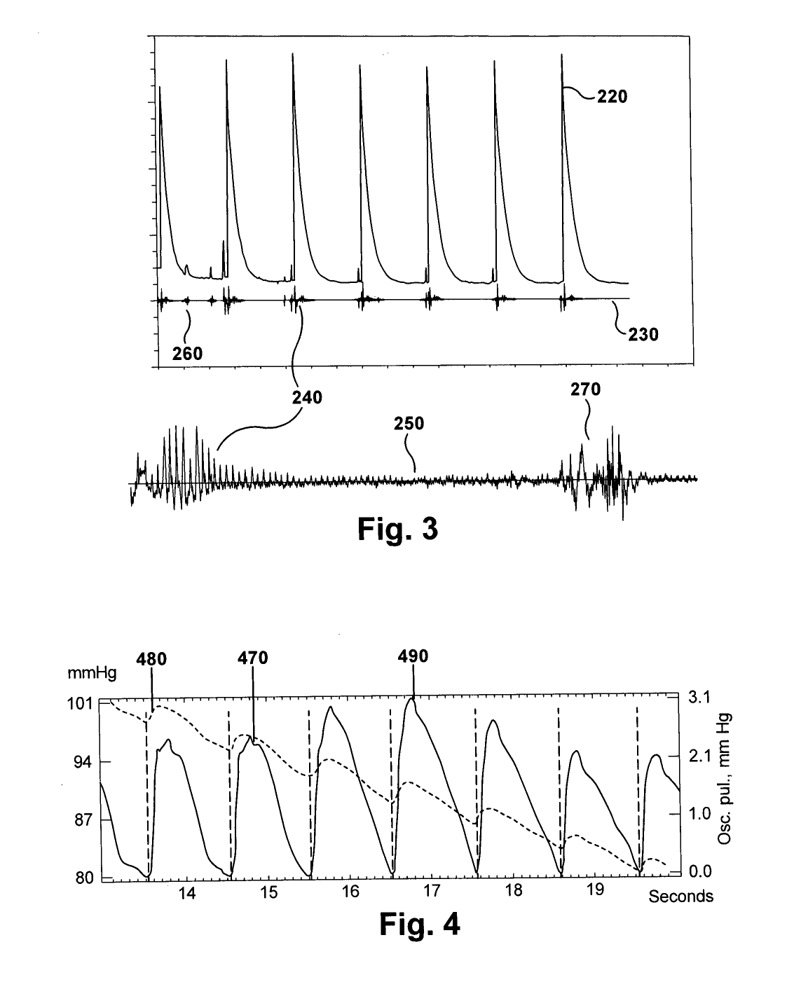

FIG. 3 provides an example of the raw signals 220 obtained by the transducer from the fluid-filled bladder placed on a patient's wrist adjacent the radial artery, i.e., in close proximity to the artery and in physical contact with the patient's wrist, and held in place by a pressurizable cuff. After the signal has been filtered through a high band pass filter, the signal 230 is used by the controller to calculate the BP and scale the pulse signals between oscillometric measurements. An expanded view of one of these filtered signal periods 230 is shown in 240 which demonstrate the envelope of maximum pulse amplitudes around the Mean Arterial Pressure and the lower signals of the pressure pulse amplitudes 250 obtained by the bladder at reduced pressures. The signal processing must be capable of identifying the true pulse signals from the noise and external disruptions 270.

Since the fluid-filled bladder is sandwiched between the patient and the pressure cuff, it will detect both the pressure exerted by the inflated cuff as well as the oscillations coming from the blood pressure pulses in the artery. FIG. 4 shows results of a session where seven consecutive measurements of the blood pressure took place, every three minutes.

FIG. 4 depicts a window of seven BP pulses where the peak amplitude pulse 490 is in the center. It shows both the decaying components of the air pressure 480 during deflation of the cuff and the oscillatory pulses 470 detected by the bladder transducer or an air-pressure pulse transducer. The oscillatory pulses marked as 470 are the vibration after de-trending. It shows the pulses after removing the decaying component or the trend (DC). There are different ways of separating the DC component from the oscillatory component. Usually, this is done by high-pass filtering or differentiation but this might affect the pulse shape.

Both the amplitude and the change in the BP signal shape can be seen clearly. The central pulse of the oscillatory pulses marked as 490 is the one that has the highest amplitude and therefore marks the peak that corresponds to the mean arterial pressure.

The graph shows the oscillations only, after the upper signal has been high pass filtered with a 4^{th}-order Butterworth IIR filter with 0.003 normalized cutoff frequency.

Looking at this resulting signal reveals some interesting features:

1. there is a clear peak to the oscillatory distribution that corresponds to the mean arterial pressure;

2. there is a clear change in pulse shape before and after the peak, which feature can be used to improve noise control and to provide a more robust identification of the peak and the mean arterial pressure;

3. the systolic pressure is approximately 60% of the peak amplitude in front of the mean arterial pressure point;

4. the diastolic pressure is approximately 80% of the peak amplitude on the other side of the mean arterial pressure point;

5. even when the pressure of the cuff drops to zero between measurements, the cuff holds the bladder against the artery sufficiently that the blood pressure signal is still observable; and

6. in lower signal-to-noise regions, the waveform must be reconstructed and artifacts must be removed to obtain a useful signal.

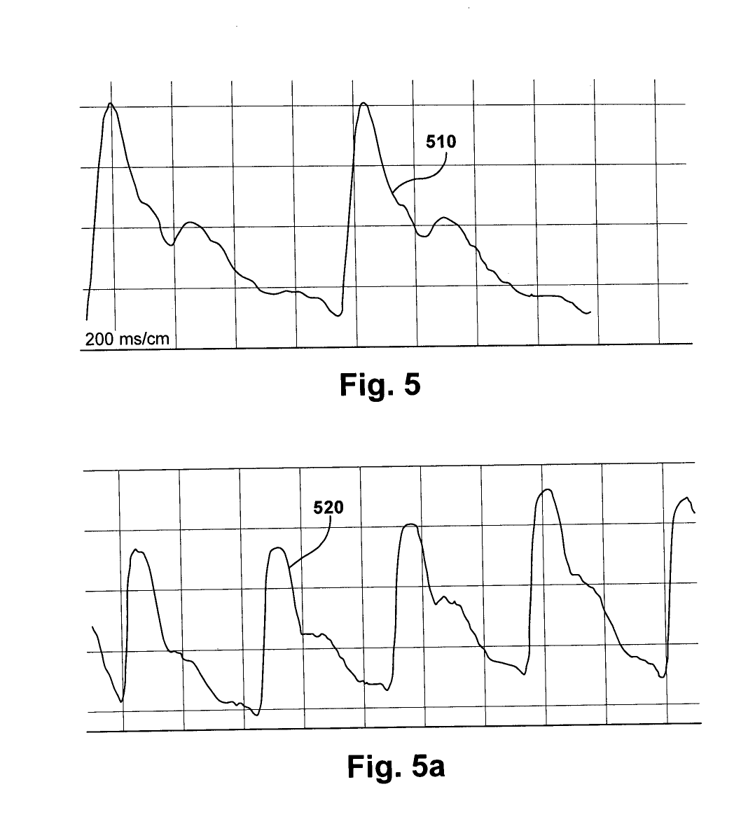

Another phenomenon that can be seen in FIG. 4, and even more in FIG. 5 and FIG. 5A is the change in the pulse shape above (510) and below (520) the mean arterial pressure. FIG. 5 shows the change in the BP signal shape, when the external pressure is above the systolic pressure to the shape of the BP signal (FIG. 5) and when the external pressure is below the diastolic pressure (FIG. 5A).

### Signal Processing

The scaling adapter controller processes the signal, removing noise and effectively increasing the sensitivity of the pressure transducer for measuring the blood pressure and scaling the blood pressure signal accordingly. The scaling adapter also operates on the pressure signal from the bladder, as sensed by the transducer, and scales the signal to fit between calculated systolic and diastolic pressure.

### Peak Detection

Since we are working with a system that has low signal-to-noise ratio, one of the most important steps is to identify each BP pulse signal. This is done by employing a peak detector operation on the raw data from the bladder transducer.

Only pulses that qualify as blood pressure signals and not motion artifacts are used. When the pressure in the cuff drops to ambient pressure, the transducer detects the internal pressure of the bladder alone. This pressure generally is below 50 mmHg and typically about 20-30 mmHg. At this low pressure the signal-to-noise ratio is low but good enough to detect a blood pressure signal from the artery. In this low signal-to-noise environment, the blood pressure signals have to be reconstructed and noise removed to provide a good picture of the blood pressure waveform.

### PCM-100

The PCM-100 embodiment includes the same elements as the PCM-50 system just described, but the air-pressurizable cuff and related components are incorporated into the PCM-100 system rather than being provided separately. The PCM-100 system also controls the air pressure in the pressure cuff. Specifically, the PCM-100 system includes both a fluid-filled bladder and an air-pressurized cuff, with a scaling adapter controller that controls and monitors the pressure in both the bladder and the cuff. This embodiment allows for a feedback loop between the BP pulse signal obtained through the bladder and the initiation of air-cuff inflation for oscillometric measurements, or for increasing or decreasing the pressure in the bladder between oscillometric measurements, thereby improving the pulse signal or patient comfort or a combination thereof.

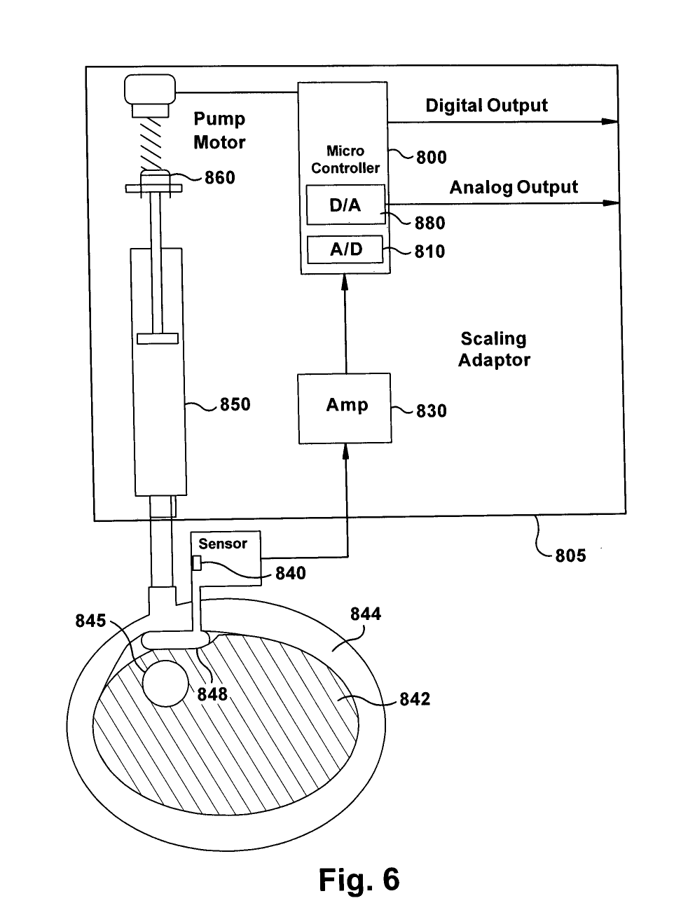

FIG. 6 illustrates an embodiment of the PCM-100 system. A processor 800 inside the scaling adapter controller 805 provides means for controlling the air-pump in the pressure cuff component 844. The processor 800 also reads the pressure from the pressure transducer 840 through an amplifier 830 and an analog-to-digital converter 810 as part of a control loop for tracking the MAP.

The scaling adapter for the PCM-100 system includes controls for the pump (such as a syringe pump, geromotor, or other pumping system that can increase and decrease pressure), motor 860, and pump 850. The illustrated scaling adapter also includes an analog-to-digital converter 810 that converts the analog signal from the pressure transducer 840 into a digital signal. The transducer 840 measures the pressure in the bladder 848, which is positioned to palpate the BP pulse signal over the artery 845 in body part 842. The bladder, which is preferably filled with an incompressible fluid, is pressed against the artery 845 by inflating the pressure cuff 844, typically with air.

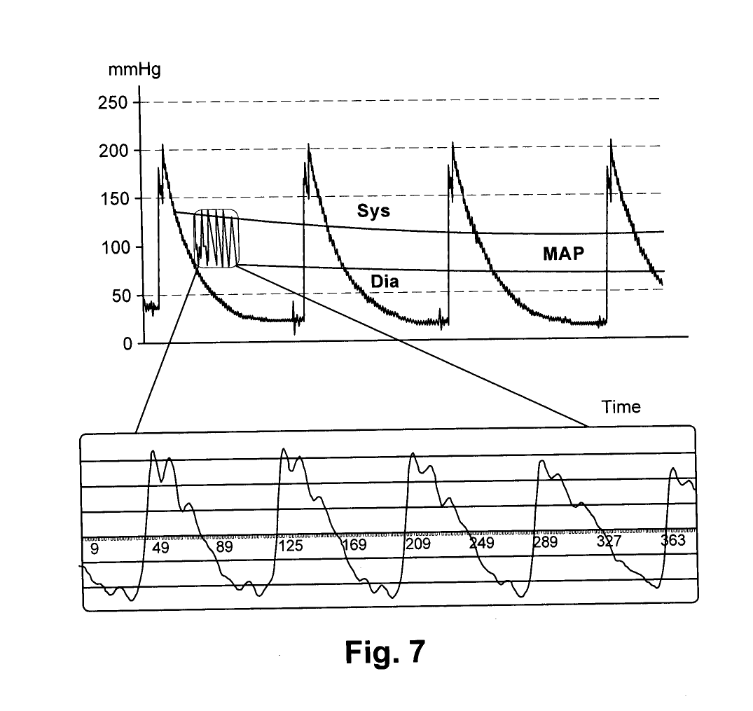

In addition to the controller's role as a continuous BP measurement device, it can also scale the BP signals of the fluid-pressure transducer 840 to the range expected from an invasive A-line and transmit an analog output signal through a digital-to-analog converter 880. This scaling will be further explained with reference to FIG. 7.

FIG. 7 describes the changes in the signals produced by the scaling adapter, in order to emulate an A-line transducer output to a standard monitor.

An illustration of the input, intermediate and output signals for a scaling adapter using the measurement system described above, using a transducer sensitivity of 5 μV/V/mmHg, is presented in FIG. 7. FIG. 7 shows a calibrated signal with a systolic pressure of 120 mmHg and a diastolic pressure of 80 mmHg for the first beat of the waveform (FIG. 7, top). The corresponding equivalent differential A-line transducer output voltage for the systolic pressure is given by (120 mmHg*5 V*5 μV/V/mmHg), or 3,000 μV. Similarly, the corresponding equivalent differential A-line transducer output voltage for the diastolic pressure is given by (80 mmHg*5 V*5 μV/V/mmHg), or 2,000 μV.

The scaling adapter effectively scales the BP signal, obtained noninvasively, in such a way that the A-line monitor sees it as if it were a regular A-line transducer from a fluid-filled pressure monitoring line (FIG. 7, bottom). It converts the BP signal obtained from the bladder into the signal that would have been produced by an indwelling cannula in an A-line system.

To scale to the absolute BP, the scaling adapter generalizes the oscillometric method to measure mean arterial pressure continuously.

Our continuous oscillometric BP measurement includes moving such a window along the oscillatory (and absolute) pulses, searching for the peak. We start with an external pressure of the bladder that is below the diastolic BP and increase the pressure in the pressure cuff until we reach the peak. Once the peak is reached and the window is centered on it, signal processing software, employing fuzzy logic control, for example, can be used to track the peak over time (which corresponds to the mean arterial pressure).

From time to time, there is a wider sweep in pressure of the air cuff, to estimate the shape of the oscillatory pulse distribution around the peak. This is done to estimate systolic and diastolic pressure, as presented in principle in FIG. 1. It is assumed that the oscillatory pulse distribution does not change as frequently as the MAP, and therefore can be sampled less frequently.

As described above, the role of the controller is to implement the method of continuous BP measurement and to perform the signal analysis, including wavelets de-noising filtering and wave analysis, for the tracking the mean arterial pressure.

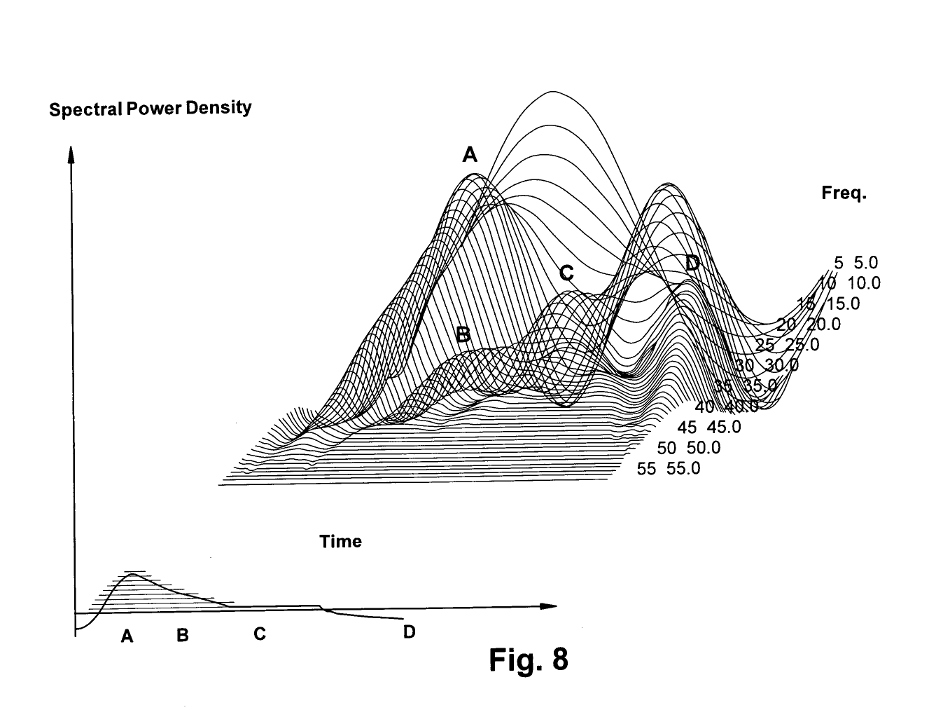

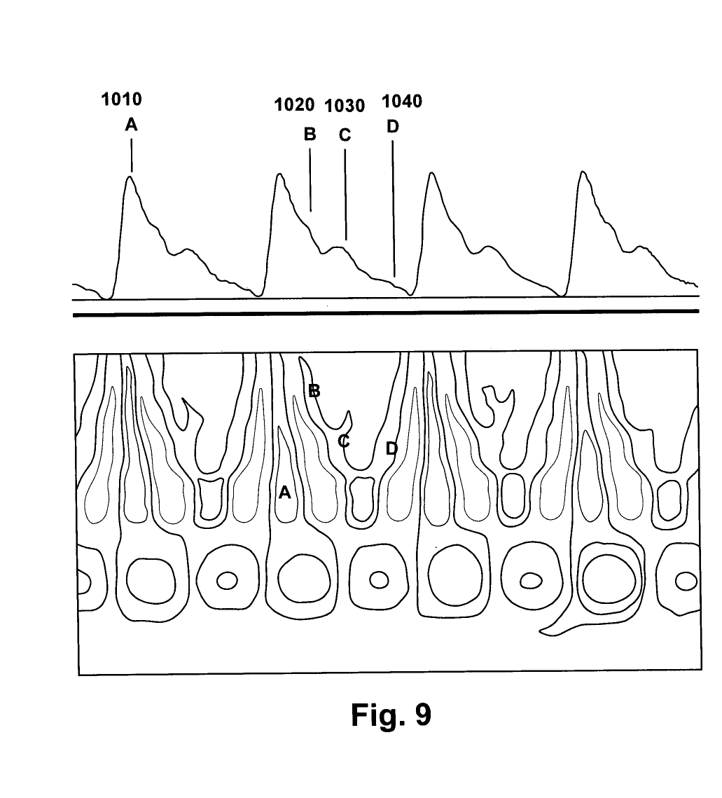

Although the cuff assembly is simple, and the oscillatory method is known and implemented in almost all automated BP monitors, it requires some sophisticated signal processing to ensure proper and reliable operation. FIGS. 8 and 9 illustrate wavelets analysis for filtering noise from the bladder transducer signal and BP signal wave analysis.

Wavelets are used to decompose the BP signal to sub-components different in time position and frequency, as depicted in the FIG. 8. The graph in FIG. 8 plots the power spectra on the Z-axis in a 3-D plot, where the X-axis and the Y-axis are frequency and time, respectively. The figure shows the result of wavelet analysis with the Morlet (Real) mother wavelet. The resolution level is 6, and subdivision level is 16, the display is logarithmic. The signal on top is the recorded pulse wave from the bladder transducer. Four peaks were revealed, marked as A, B, C and D.

Similar results were obtained when using Gabor wavelets in FIG. 9. A, B, C and D are marked in FIGS. 9 as 1010, 1020, 1030 and 1040 respectively. From the tree diagram in FIG. 9 it looks like the peak of A belongs to a different source than peaks B, C and D that belong to a same tree. Therefore, unlike peak A, it looks like peaks B, C and D have the same origin. This corresponds to the theory according to which peak A represents the main BP signal wave coming from the heart, while B, C and D are the reflected waves. B is considered to be the reflection from the renal artery's bifurcation while C is the reflection from the Iliac bifurcation. D is considered to be 2nd harmonics. In clinical medicine, from the ratio between the peaks of A and B, the Augmentation Index is computed and it is used as an indication of arteriosclerosis.

### Tracking the MAP

The tracking of the mean arterial pressure (MAP) is an objective of the scaling adapter controller. By measuring the BP in the oscillometric method, and extending it to continuous measurement of the MAP, the scaling adapter can scale the sensed BP signal to its true values in mmHg, even at low air-cuff pressures.

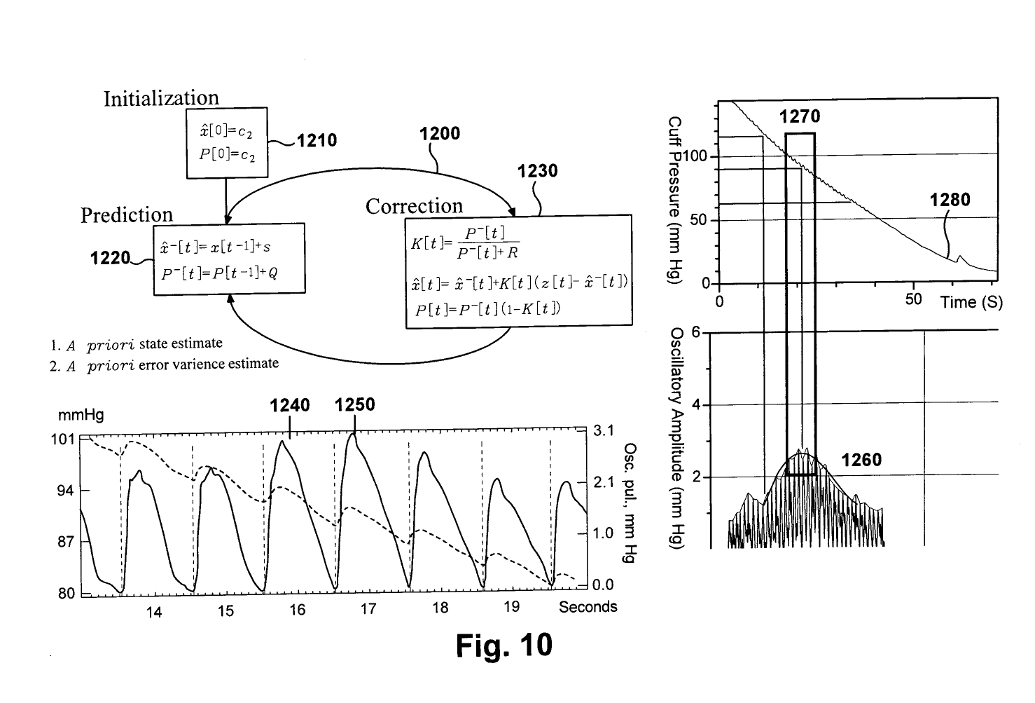

This might look straightforward, as the oscillometric method stops the decaying pressure when the peak oscillatory pulse is detected (which corresponds to the MAP location on the decaying pressure curve). Then the pressure oscillates around the MAP in a narrow window as depicted in 1240 in FIG. 10. The window 1240 shows a drop in pressure between 101 mmHg and 80 mmHg over seven seconds. The pulse that is marked as 1250 is the one with the peak amplitude. It corresponds to a MAP of about 92 mmHg.

Suppose that the real MAP will increase now to 97 mmHg. This will move the highest amplitude pulse two pulses to the left, and all we need to do is to move the sweep to be between 106 and 85 to keep our peak pulse in the center of the window.

In reality, this is not that simple as the measurement of the pulse's amplitude involves some fluctuations due to noise and error in measurement. As we can see in FIG. 10 in the oscillatory pattern 1260, there are several random peaks, and just following the highest amplitude pulse can throw us away from the tracking of the MAP.

Therefore, we need a tracking algorithm that can track the position of the peak (and from it the value of the MAP) in a reliable way.

We can formulate it as a control problem of tracking the position of the peak (which corresponds to the MAP), where the input is the pulse position x, and the output is a voltage that corresponds to the pressure at the air-pump (or valve if we want to decrease the pressure). There are several ways to implement the tracking of the position of the peak that corresponds to the MAP.

The simplest one increases the cuff pressure to the point where maximal oscillations are detected. This point corresponds to the mean arterial pressure.

The goal of the system now is to track the MAP by following it. In FIGS. 1 and 4, we can see how the oscillations reach the maximal point. In FIG. 4 we can see a window in which the center wave has the highest amplitude. Another phenomenon that has been observed by the author of this patent is that the pulse shape changes when the external pressure is higher or lower than the MAP, as in FIGS. 5 and 5A. By using wavelet analysis, as shown in FIGS. 8 and 9, we can determine the change in shape of the BP signal above and below MAP.

We can use both the change in amplitude and change in the pulse shape, as measured by the wavelet decomposition, to track the MAP. This is done by increasing or decreasing the external pressure to keep the transmural pressure as closed to zero as possible.

Since maintaining cuff pressure equivalent to the MAP can be uncomfortable for the patient if applied for long period, we drop the cuff pressure to a lower pressure, and continue to monitor the pulse amplitude at this level for certain time or until a BP change threshold is exceeded.

Another implementation is by oscillating the external pressure provided by the air cuff in a narrow pressure range 650 around the MAP, as depicted in FIG. 1 In this case, the external pressure is dropped to 10 mmHg after 3 minutes or less, to avoid impeding blood flow for long periods of time.

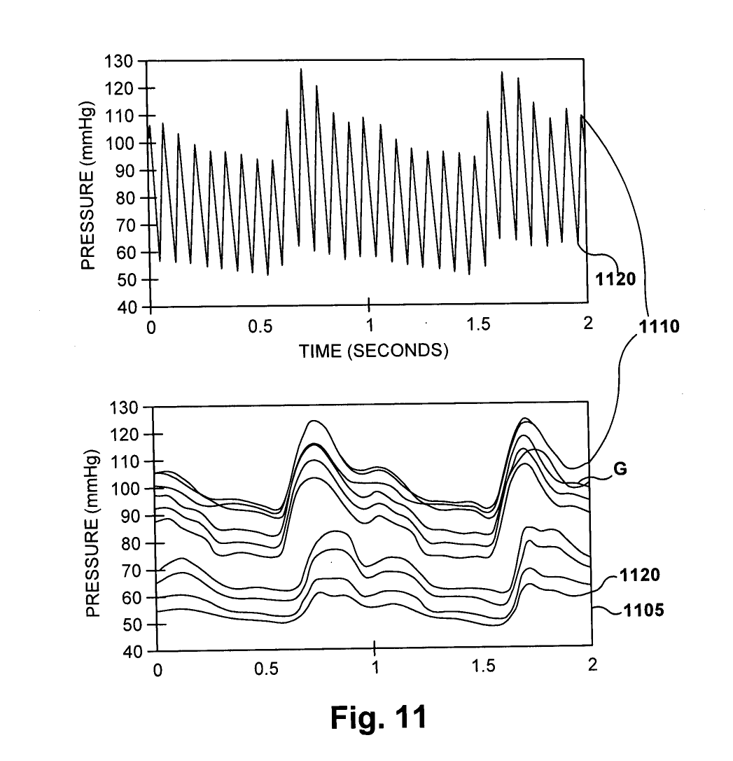

FIG. 11 depicts a third implementation, which is similar to the previous one except that the pressure exerted by the air-cuff is pulsated and changed at a higher frequency (1110). In this implementation, the vibrations (1100) that modulate the BP signal are caused by modulating the air cuff pressing pressure or by modulating the fluid-filled bladder. The pump pulse frequency is selected to be higher than the highest frequency components of interest in the artery's blood pressure waveform, and thus the pressure signal may be accurately filtered for its modulation and heartbeat components. In 1105 we can see how from the change in the amplitude corresponding to the pressure range 1110 to 1120 enables us to reconstruct the BP signal corresponding to each pressure level.

If the artery is under-compressed, then the pressure response to the pump pulse's oscillation will be larger during systole than during diastole. Conversely, if the artery is over-compressed, then the amplitude of the pump pulse's pressure oscillation will be larger during diastole than during systole. However, if the artery is optimally compressed, then the amplitudes of the pump pulses pressure oscillation during the systolic and end-diastolic stages will be substantially the same. In addition, the overall amplitude of the pressure oscillation is at a minimum when the artery is optimally compressed. With this information, regarding the under-or over-compression of the artery, the air-cuff pressure may be adjusted to the MAP.

A control system for implementing the control scheme described above is depicted in FIG. 6. The pump pulses, either of the air pump or a vibrator that modulates the fluid-filled bladder pressure can create pressure pulses that modulate the BP pulse signal as depicted in FIG. 11.

The filtered signal, which incorporates only the pump pulse component of the pressure signal after de-noising, is subjected to analysis. Analysis of the filtered signal and comparing its amplitude during systole with its amplitude during diastole, determines whether the artery is under-compressed, over-compressed, or optimally compressed. The analyzer then produces a corresponding error signal that can be used to arrive at the external pressure that corresponds to the MAP.

### Tracking Algorithms

To track the mean arterial pressure (MAP) using features of the changing location of the peak in the oscillatory distribution with changes in the MAP and the change of the pulse shape with the MAP, it is advantageous to employ a robust and efficient tracking algorithm.

Tracking is the key factor in adaptive algorithms of all kinds. The most familiar general family of tracking algorithms for linear regression models includes the familiar LMS (Least Mean Squares or gradient approach), RLS (recursive least squares) and KF (Kalman filter)-based estimators.

A Kalman filter provides the following advantages:

(a) progressive least-squares;

(b) only two steps: prediction and filtering;

(c) high fidelity prediction: rejects noise efficiently;

(d) faster filtering, much smaller matrix to be inverted than in the global model; and

(e) elegantly accommodates process noise., e.g. motion artifacts, without introducing correlated errors.

The rudimentary discrete scalar Kalman filter that we described in FIG. 10 is a recursive Markov process, where every new step depends on the previous step alone. The algorithm can be divided into three major parts as depicted in FIG. 10 in the Kalman Filter control loop marked as 1200:

Initialization—marked as 1210. The initialization step provides the initial values for x, the position of the peak pulse and P, the error variance. The first estimate is done from the initial BP measurement that starts the process.

Prediction—marked as 1220. This is the a-priori state estimate and the a-priori error variance estimate at time t, as calculated from the time t−1.

Correction—marked as 1230. This is the calculation of the filter gain—K, the a-posteriori state estimate (under the conditions of the measurement) and the a-posteriori error variance estimate.

The first oscillometric measurement of the BP provides good initialization of the process variables and helps to increase the convergence speed of the process noise variance. The prediction step is very useful for forecasting the next system state, especially in the case of slow system updates by full oscillometric measurements. The correction step incorporates the measured values and adapts the Kalman filter process variables to the best verification with reality.

The exact expressions for the quality of the obtained estimates are complicated. Approximate, and easy-to-use, expressions for the covariance matrix of the parameter tracking error have been developed. These are applicable over a large time interval, including the transient and the approximation error, and can be explicitly calculated. As shown in FIG. 10, a Kalman filter is very effective in performing a tracking task.

Another implementation, however, uses a fuzzy logic controller in addition to or in place of the Kalman filter. One advantage is that fuzzy logic controllers can provide a similar result with fewer steps and simpler computation, using look-up tables for the membership functions. This can allow the use of a smaller and less expensive processor and related components for the controller.

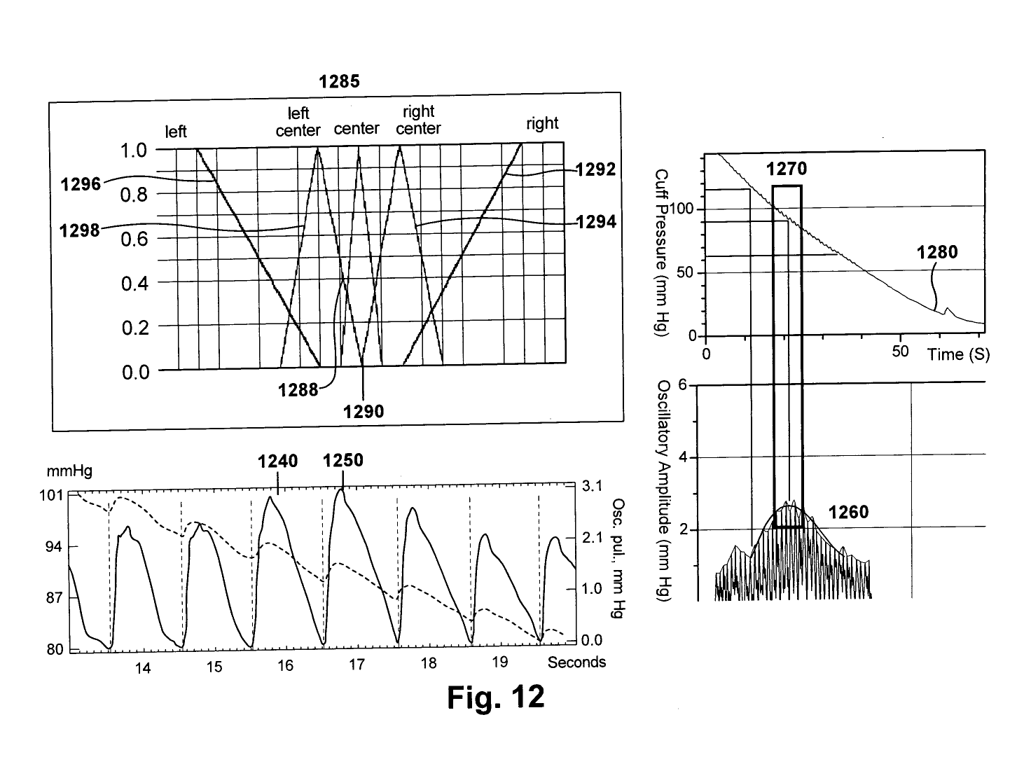

In FIG. 12, part 1285 shows five linguistic variables (Left—1296, Left Center—1298, Center—1290, Right Center—1294, Right—1292) with their respective membership functions. The position refers to the position of the peak pulse 1250 in window 1240 as depicted below. This window is located on the decaying slope of the cuff pressure. This means that being right of the center corresponds to a cuff pressure that is lower than the MAP, while being left of the center corresponds to a cuff pressure that is higher than the MAP.

The use of the linguistic variables provides the fuzzification of the system, the implementation of fuzzy logic signal analysis to track the MAP. Fuzzy logic can be viewed as a series of IF-THEN rules. These rules express the relationship between certain positions' memberships in each of the fuzzy sets, for example five fuzzy sets, and the control of the pump.

The fuzzy inference process results in a linguistic value for the output variable. In our example, let's look at a position marked by 1288, which is a bit to the left of the center. Position 1288 has zero membership in the variable Left, 0.4 membership in the variable Left Center, 0.1 membership in the variable Center, and zero membership in variables Right Center and Right. We can interpret the linguistic value {0.0, 0.4,0.1, 0.0, 0.0} as just slightly left. To use this linguistic value to adjust the pressure in the cuff by controlling the air pump, it must be translated into a real (physical) value. This step is called defuzzification. The pump can then be operated to increase the cuff pressure and move the window to the left so the peak is in the center of the window.

The relationship between the linguistic values and the corresponding real values always is given by the membership function definitions describing the terms of the linguistic output variable (see FIG. 12, part 1285).

In our example, we obtained a fuzzy inference result that is both fuzzy and ambiguous because there are two different actions with nonzero truth degrees to be taken at the same time. One is Center, which means do nothing, and the other one is Left Center which means operate the pump. In a fuzzy controller you must combine two conflicting actions that are defined as fuzzy sets to form a crisp real value. A solution to this problem is to find the best compromise between the two different goals. This compromise represents the best final conclusion received from the fuzzy inference process.

To complete this presentation, the reader should note that detection of the peak amplitude that corresponds to MAP can be done in two ways. One way is to increase the pressure from below MAP to above MAP. The other way is the most common way of decaying pressure. If detection is done both when increasing and decreasing it makes it more efficient.

The other thing to note is that the pump that we mention is bidirectional to allow both increase and decrease of pressure. Alternatively, we can use a unidirectional pump to increase the pressure and a valve to release pressure. Another other possibility with a unidirectional pump is to change valve direction or to use two pumps: one for pumping air in and one for pumping air out.

### General Operation of the Scaling Adapter

A schematic presentation of a continuous oscillometric monitoring system in accordance with the invention is shown in FIG. 2. FIG. 2 shows the main elements of an inflatable cuff of a non-invasive blood pressure monitor and the fluid-filled bladder, fluid pressure transducer, scaling adapter controller, and an output device, such as a vital signs monitor.

In general, operation of the scaling adapter (see FIG. 6) involves the following steps.

a) Step 1. When connected to a display, such as a vital signs monitor, The scaling adapter controller starts reading the excitation voltage supplied by the monitor, and passes the A-line sensor or bladder transducer output through an analog-to-digital converter (ADC).

b) Step 2. The controller increases the pressure in the cuff using the air-pump, until the signal from the A-line sensor reaches its peak.

c) Step 3. Through the unloading method, where the air pump is controlled in response to tracking the MAP, the controller measures continuously mean arterial pressure in mmHg.

d) Step 4. The controller controls the air pump to track the peak pressure and continuously estimates systolic and diastolic pressure. The search window is depicted in FIG. 4. Tracking is based both on amplitude (FIGS. 4—480) and change in BP signal shape (see FIGS. 4—470). FIGS. 4—490 shows the peak amplitude of the oscillatory vibration which corresponds to the MAP in the center of the moving window.

e) Step 5. In parallel with step 4, the controller scales the pulse sensor signal according to systolic and diastolic points to emulate an invasive A-line.

f) Step 6. After a predetermined time, such as three minutes, cuff pressure is dropped to a predetermined pressure below the MAP and tracking continues for the MAP.

g) Step 7. After a predetermined time or a change in pressure outside a predetermined range, go back to step 2 and repeat.

For the scaling adapter to be able to output the correct output signal to the output monitor for any measured blood pressure, the controller needs to know in advance the transducer sensitivity with which the monitor is configured to work, as well as the excitation voltage supplied by the monitor. The excitation voltage is supplied by the monitor and sensed by the controller through an excitation signal conditioner. The transducer sensitivity needs to be provided to the scaling adapter by the user, however, and it must be the same as that of the transducer sensitivity which the IBP monitor is configured to work with. This sensitivity is normally specified in the monitor's manual.

The scaling adapter can be designed in such a way that the transducer sensitivity is selectable by the user. Additionally, it can be designed to operate on a default sensitivity of 5 ·mu·V/V/mmHg, the most commonly used sensitivity, if no selection is made.

To improve the smoothness of the blood pressure waveform at the output monitor's end, the resolution of the low-glitch DAC output voltage in LSB per volt should be maximized in such a way that the DAC is still able to produce the required DAC output voltage range, depends on the transducer sensitivity, excitation voltage, pressure measurement range, and the scaling factor for the DAC output voltage. All this can be accomplished by using a programmable DAC that allows its full-scale output voltage range to be configured by the controller, and by configuring the DAC for a full-scale output voltage range that is slightly larger than the required DAC output voltage range.

To further improve the smoothness of the blood pressure waveform that is displayed on the monitor, the number of the digital values for the waveform can be increased by interpolation. A simple method is to perform linear interpolation between every two adjacent data points. Nonlinear interpolation methods such as quadratic interpolation and cubic spline interpolation can also be used.

For a full emulation of the output voltage level of the IBP transducer, the equivalent IBP transducer output voltage produced by the Scaling Adaptor should be such that the voltage level of each of the two terminals for this equivalent voltage is the same as the level that would be produced at the corresponding output terminal of the transducer. Since the nominal midpoint voltage of the IBP transducer output signal is the same as the midpoint voltage between the excitation terminals, as mentioned above, this emulation can be accomplished by centering the differential output voltage about the midpoint voltage between the excitation terminals. In other words, the midpoint of the differential output voltage rides on the midpoint voltage between the excitation terminals, or the midpoint of the differential output voltage is offset with respect to the negative excitation terminal E− by half the voltage across the excitation terminals.

An approximate emulation of the A-line transducer output voltage level can be achieved by making one of the terminals for the differential output voltage take on the midpoint voltage between the excitation terminals. This approximate emulation is judged to be adequate because the differential output voltage is relatively small, being usually in the order of millivolts or tens of millivolts, compared to the midpoint voltage between the excitation terminals, which is of usually in the order of volts as measured with respect to the negative excitation terminal E−. Additionally, the circuitry for implementing this approximate emulation is likely to be simpler than that for the full emulation.

A-line monitors use the input impedance, output impedance, or both to detect the presence and absence of a transducer or whether the transducer is functioning properly, so these impedances should be emulated. Emulating these impedances will more accurately emulate the actual situation and help to reduce the chances of problems in communication between the scaling adapter and A-line monitor.

Zeroing the scaling adapter to the A-line monitor can be easily performed in a way that is similar to that for a fluid-filled invasive A-line system.

The scaling adapter according to the present invention typically operates based on a known A-line transducer sensitivity, accepts the excitation voltage provided by the A-line monitor, and produces an equivalent A-line transducer output signal corresponding to the measured blood pressure. The scaling adapter also emulates the input and output impedances of the A-line transducer with which the A-line monitor is configured to work. The A-line monitor itself may be connected to a central monitoring system, but this connection may not be essential.

The disclosed apparatus offers the advantage of enabling continuous beat-to-beat blood pressure to be monitored by noninvasive means, while allowing the medical staff to follow the same work flow and continue to use existing A-line monitors with which they are already familiar. It allows medical staff to continue to benefit from multi-parameter monitoring offered by patient monitors that provide monitoring of vital signs such as ECG, oxygen saturation, respiration, and cardiac output, in addition to blood pressure. It also allows them to continue to benefit from the use of the central monitoring system to which the A-line transducer or patient monitors are connected.

Zeroing of A-Line Transducer with A-Line Monitor

The output voltage of an A-line transducer at zero mmHg is usually not zero. This output voltage is called the zero offset or zero balance. This offset voltage is sometimes augmented by hydrostatic pressure caused by a column of fluid above the level of the sensing area of the transducer. For accurate blood pressure measurement, the transducer must be zeroed with the monitor before monitoring begins. During the zeroing of the transducer, the monitor effectively reads the total offset voltage and associates it with zero mmHg, and in doing so, establishes a zero-mmHg reference level for the monitor.

The zeroing procedure for A-line monitoring system requires the clinician to manually trigger the monitor to perform the zeroing. It includes the following steps: a) prepare the A-line monitor to receive the transducer output voltage at zero mmHg; b) position the zeroing port of the transducer so that it is at the patient's mid-heart level; c) turn the handle of the zeroing stopcock OFF and loosen or remove the dead-ender cap on the zeroing side port. This step blocks the fluid pressure transducer from the bladder pressure and opens the air pressure transducer to the atmosphere. Some fluid will flow out of the side port as a result. Then in step d) the clinician will zero the transducer with the A-line monitor by pressing the appropriate key or button on the monitor. This zeroing has to be activated manually because there is no automated feedback to check whether or not the fluid-filled system is ready to be zeroed.

In our proposed system this is not necessary, however, as it does not need the process of zeroing. The non-invasive blood pressure monitoring system provided by the invention calibrates the continuous BP signal and therefore any offset is automatically compensated for after the first measurement.

The common denominator of existing BP measurements is either Intermittent oscillometric measurements or continuous tonometric measurement. When combined, it is starting from the tonometric paradigm and calibrating by the oscillometric method.

In contrast, we start from an oscillometric method and apparatus, and extend it to the continuous case by time-based estimation of the BP signal between consecutive oscillometric measurements.

For achieving this, we use the following:

a. increasing sensitivity by using a fluid-filled bladder so we can work on the lower part of the oscillatory pulse distribution (well below diastolic in contrast to tonometry that works around MAP);

b. learning the BP signal around MAP and decomposing it to its components using wavelets, employing the learned template for peak detection in low signal-to-noise environments at low cuff pressures; and

c. employing intermittent oscillometric measurements, a model, and a Kalman filter to reconstruct the BP signal at low cuff pressures and scale it to the absolute values measured at higher cuff pressures.

Below, we elaborate on these three methods:

a. Increasing Sensitivity

Increasing sensitivity of the inflated cuff in the regular oscillometric monitor is achieved by using a fluid-filled bladder placed under the inflatable cuff and adjacent an artery. This fluid-filled bladder is maintained at a fixed pressure below the diastolic pressure (e.g. 50 mmHg).

Since the bladder is filled with fluid that is incompressible, it is much more sensitive to the arterial pulse and propagates the pulse through fluid-filled tubing to the pressure transducer. The fluid-filled bladder detects the oscillometric vibrations during both inflation and deflation, and the BP signal between two consecutive inflation phases of the oscillometric method.

Using advanced signal processing, that will be described below, we can work on the lower part of the oscillatory pulse distribution (well below diastolic in contrast to tonometry that works around MAP).

b. Signal Processing

The major goal of the signal processing is to analyze the fluid-filled bladder pressure signal that is influenced both by the pressurizable cuff and by the arterial pulse. Systolic, diastolic and the MAP are computed. The main challenge is to restore the BP signal and calibrate it against a standard. For that, there is a need to detect the BP signals and differentiate between legitimate BP signals and noise. For performing this task, we take advantage of the fact that the individual pulse shape is shaped by the individual, and unique cardiac and arterial structure and function. A learning algorithm is employed to identify the BP signal shape around the MAP and to decompose it to its components using wavelet techniques. Then, we apply the learned template to peak detection in low signal-to-noise segments between cuff inflations.

c. Integration by Tracking Algorithms

Integrating the intermittent oscillometric measurements, a model, and a Kalman filter to reconstruct the BP signal and scale it to the absolute measured values produces the reconstructed and calibrated continuous BP signal.

Intermittent oscillometric measurements detected by the fluid-filled bladder provide calibration points every 3-30 minutes, for example, according to a user setting determined by clinical requirements.

The model of the cardiovascular system, that is identified and learned, provides the template for the individual BP signal shape and its changes across one or more wavelet components, according to treatment or physical activity. The model helps to filter out abrupt changes that could not result from the physiology or BP signal shapes that do not fit the patient (e.g. an old person cannot have suddenly a BP signal contour of a young person). Changes due to exercise or drugs have their typical rate of change (e.g. according to the drug kinetics)

The Kalman filter helps to integrate the model with the data and minimize noise. Unlike regular intermittent BP measurements, where each measurement is independent of the previous measurements, the Kalman filter takes into account previous measurements and results in optimal tracking of BP over time.

In summary, the present invention provides a method that extends the oscillometric method, which currently is used for blood pressure measurement at one point in time, to provide continuous measurement of blood pressure (BP). The method provides a BP signal that is similar to an invasive arterial line continuous BP measurement with minimal changes in clinical procedures. The apparatus for performing the method includes a sensor with a fluid-filled, disposable, flexible bladder underneath a non-invasive inflatable cuff monitor. The inflatable cuff monitor provides a single-point BP value in a traditional manner. An electronic scaling adapter estimates the diastolic pressure and systolic pressure corresponding to the BP signal obtained from the fluid-filled bladder, uses the single-point BP value from the inflatable cuff monitor to scale the BP signal detected by the bladder, and outputs a scaled BP signal that can be displayed from a conventional vital signs monitor.

Paraphrasing the claims, the present invention can be defined to include one or more of the features set forth in the following clauses:

A. A non-invasive blood pressure apparatus for use with an air-pressurizable cuff that provides a near-continuous blood pressure (BP) signal output, the apparatus comprises: a fluid-filled bladder positionable under an air-pressurizable cuff, a pressure transducer coupled to the bladder through a fluid-filled line, and a controller in electrical communication with the transducer to translate nearly continuously the pressure signal from the transducer into a BP value.

B. The apparatus of clause A or any other clause depending from clause A, further comprising a monitor and means for inputting a BP value determined from the oscillometric method using an air-pressurizable cuff, the controller being configured to scale and output the near-continuous BP signal to the oscillometrically-measured BP in real time.

C. The apparatus of clause B or any other clause depending from clause B, where the scaling adapter comprises:

i) at least one microcontroller that includes an ADC and an DAC;

ii) at least one air pump that communicates with the inflated baldder;

iii) at least one mini-pump that communicates with the fluid pressure in the bladder; and

iv) at least one algorithm that controls the air pump and determines the mean arterial pressure and pulse pressure and scales an output signal accordingly.

D. The apparatus of clause A or any other clause depending from clause A, where the pressure signal from the transducer is processed to filter out any signals that do not correlate with the subject BP signal shape.

E. The apparatus of clause A or any other clause that depends from clause A, further comprising an air-pressurizable cuff and a transducer coupled to the cuff to output the air pressure in the cuff to the controller.

F. The apparatus of clause E or any other clause that depends from clause E, where an absolute BP value is derived by employing the oscillometric method to the fluid-filled bladder pressure when pressed by inflating the air-pressurizable cuff.

G. The apparatus of clause E or any other clause that depends from clause E, where the controller is connected to the pump and the air-pressure transducer to monitor and control the pressure in the air cuff so that the air-pressure transducer signal during an inflation phase can be used to measure BP, which provides an ability to measure at least one of systolic, mean arterial pressure (MAP), and diastolic pressure in either an inflation phase or a deflation phase.

H. The apparatus of clause A or any other clause depending from clause A, where the controller includes a scaling adapter electrically connected between the bladder transducer and a vital signs monitor.

I. The apparatus of clause A or any other clause depending from clause A, where the air-pressurizable cuff is provided by a commercially-available NIBP device.

J. The apparatus of clause A or any other clause depending from clause A, where the bladder includes a diaphragm having a thickness less than 100 microns.

K. The apparatus of clause J or any other clause depending from clause J, where the diaphragm is made of a silicone rubber or a polyurethane.

L. The apparatus of clause A or any other clause depending from clause A, where the bladder is disposable.

M. The apparatus of clause A or any other clause depending from clause A, where the bladder is attached with an adhesive rim.

N. The apparatus of clause A or any other clause depending from clause A, where the bladder is attached with an adhesive bandage.

O. A non-invasive blood pressure apparatus includes a controller configured to manipulate a pressure at which a sensing bladder is pressed against the patient in proximity to a palpable artery, where the controller includes computational means configured to determine at least one of a systolic and a diastolic pressure and to scale a blood pressure signal accordingly.

P. The apparatus of clause O or any other clause depending from clause O, where the controller includes means for pumping air into the sensing bladder.

Q. The apparatus of clause O or any other clause depending from clause O, where the controller scaling adapter comprises:

i) means for pumping a fluid into at least one bladder;

ii) pressure-determining means for measuring fluid inside the bladder; and

iii) pressure-controlling means for controlling the pressure inside the bladder.

R. The apparatus of clause Q or any other clause depending from clause Q, where the pumping means comprises a syringe pump.

S. The apparatus of clause Q or any other clause depending from clause Q, where the pumping means comprises a pump selected from a group consisting of: a gear pump, a peristaltic pump, and a geromotor pump.

T. A controller configured to connect between a fluid-filled bladder that can be placed on a patient's skin in proximity to an artery, a pressure sensor, and a vital signs monitor, the controller includes:

a first input port configured to receive a signal indicative of a BP signal of a subject;

a processor configured to receive the signal and to control a fluid pump to manipulate bladder pressure and determine diastolic and systolic BP and to scale an output signal indicative of the BP of the subject patient according to a predetermined algorithm based on the oscillometric method; and

an output port configured to provide the output signal in a form suitable for input to a monitor.

U. The scaling adapter of clause T or any other clause depending from clause T, where the controller is configured to enable a standard vital signs monitor to display the scaled output signals.

V. Use of the apparatus of clause T or any other clause depending from clause T, for computing derived hemodynamic parameters like cardiac output, central BP, and systemic vascular resistance in a continuous way.

W. A method for calculating a blood pressure of a subject by manipulating the pressure of a fluid-filled bladder placed on a patient's skin in proximity to a palpable artery, comprising the following steps:

i) increasing bladder pressure and measuring a relationship between pulse amplitude and pressure change; and

ii) changing the bladder pressure in a periodic manner and estimating from a change in pulse amplitude and shape the mean arterial pressure, diastolic pressure, and systolic pressure.

X. An oscillometric BP measurement device that sweeps cuff pressure around a predetermined value below mean arterial pressure to obtain continuous measurement of the MAP.

Y. The device of clause X or any other clause depending from clause X, that uses incrementally larger cuff pressure sweeps to estimate a shape of oscillatory pulse distribution.

Z. An oscillometric BP measurement device that sweeps cuff pressure around a predetermined value below mean arterial pressure to estimate systolic and diastolic BP.

To those skilled in the art to which the invention relates, many changes in construction and widely differing embodiments and applications of the invention will suggest themselves without departing from the scope of the invention as defined in the appended claims. The disclosures and the descriptions herein are purely illustrative and are not intended to be in any sense limiting.

What is claimed is:

1. A non-invasive blood pressure apparatus for use with an air-pressurizable cuff that provides a near-continuous blood pressure (BP) signal output, the apparatus comprises: a fluid-filled bladder positionable under an air-pressurizable cuff, a pressure transducer coupled to the bladder through a fluid-filled line, and a controller in electrical communication with the transducer to translate nearly continuously the pressure signal from the transducer into a BP value.

2. The apparatus of claim 1, further comprising a monitor and means for inputting a BP value determined from the oscillometric method using an air-pressurizable cuff, the controller being configured to scale and output the near-continuous BP signal to the oscillometrically-measured BP in real time.

3. The apparatus of claim 2, where the scaling adapter comprises:
a) at least one microcontroller that includes an ADC and an DAC;
b) at least one air pump that communicates with the inflated baldder;
c) at least one mini-pump that communicates with the fluid pressure in the bladder; and
d) at least one algorithm that controls the air pump and determines the mean arterial pressure and pulse pressure and scales an output signal accordingly.

4. The apparatus of claim 1, where the pressure signal from the transducer is processed to filter out any signals that do not correlate with the subject BP signal shape.

5. The apparatus of claim 1, further comprising an air-pressurizable cuff and a transducer coupled to the cuff to output the air pressure in the cuff to the controller.

6. The apparatus of claim 5, where an absolute BP value is derived by employing the oscillometric method to the fluid-filled bladder pressure when pressed by inflating the air-pressurizable cuff.

7. The apparatus of claim 5, where the controller is connected to the pump and the air-pressure transducer to monitor and control the pressure in the air cuff so that the air-pressure transducer signal during an inflation phase can be used to measure BP, which provides an ability to measure at least one of systolic, mean arterial pressure (MAP), and diastolic pressure in either an inflation phase or a deflation phase.

8. The apparatus of claim 1, where the controller includes a scaling adapter electrically connected between the bladder transducer and a vital signs monitor.

9. The apparatus of claim 1, where the air-pressurizable cuff is provided by a commercially-available NIBP device.

10. The apparatus of claim 1, where the bladder includes a diaphragm having a thickness less than 100 microns.

11. The apparatus of claim 10, where the diaphragm is made of a silicone rubber or a polyurethane.

12. The apparatus of claim 1, where the bladder is disposable.

13. The apparatus of claim 1, where the bladder is attached with an adhesive rim.

14. The apparatus of claim 1, where the bladder is attached with an adhesive bandage.

15. A non-invasive blood pressure apparatus includes a controller configured to manipulate a pressure at which a sensing bladder is pressed against the patient in proximity to a palpable artery, where the controller includes computational means configured to determine at least one of a systolic and a diastolic pressure and to scale a blood pressure signal accordingly.

16. The apparatus of claim 15, where the controller includes means for pumping air into the sensing bladder.

17. The apparatus of claim 15, where the controller scaling adapter comprises:
a) means for pumping a fluid into at least one bladder;
b) pressure-determining means for measuring fluid inside the bladder; and
c) pressure-controlling means for controlling the pressure inside the bladder.

18. The apparatus of claim 17, where the pumping means comprises a syringe pump.

19. The apparatus of claim 17, where the pumping means comprises a pump selected from a group consisting of: a gear pump, a peristaltic pump, and a geromotor pump.

20. A controller configured to connect between a fluid-filled bladder that can be placed on a patient's skin in proximity to an artery, a pressure sensor, and a vital signs monitor, the controller includes:
a first input port configured to receive a signal indicative of a BP signal of a subject;
a processor configured to receive the signal and to control a fluid pump to manipulate bladder pressure and determine diastolic and systolic BP and to scale an output signal indicative of the BP of the subject patient according to a predetermined algorithm based on the oscillometric method; and
an output port configured to provide the output signal in a form suitable for input to a monitor.

21. The scaling adapter of claim 20, where the controller is configured to enable a standard vital signs monitor to display the scaled output signals.

22. Use of the apparatus of claim 20 for computing derived hemodynamic parameters like cardiac output, central BP, and systemic vascular resistance in a continuous way.

23. A method for calculating a blood pressure of a subject by manipulating the pressure of a fluid-filled bladder placed on a patient's skin in proximity to a palpable artery, comprising the following steps:
a) increasing bladder pressure and measuring a relationship between pulse amplitude and pressure change; and
b) changing the bladder pressure in a periodic manner and estimating from a change in pulse amplitude and shape the mean arterial pressure, diastolic pressure, and systolic pressure.

24. An oscillometric BP measurement device that sweeps cuff pressure around a predetermined value below mean arterial pressure to obtain continuous measurement of the MAP.

25. The device of claim 24 that uses incrementally larger cuff pressure sweeps to estimate a shape of oscillatory pulse distribution.

26. An oscillometric BP measurement device that sweeps cuff pressure around a predetermined value below mean arterial pressure to estimate systolic and diastolic BP.
